(19) United States
(12) Patent Application Publication
(10) Pub. No.: US 2024/0099561 A1
(43) Pub. Date: Mar. 28, 2024

(54) ENDOSCOPE CONNECTION MECHANISM, ENDOSCOPE ADAPTER, AND ENDOSCOPE SYSTEM

(71) Applicant: OLYMPUS MEDICAL SYSTEMS CORP., Tokyo (JP)

(72) Inventors: Yuichi TOGAWA, Tokyo (JP); Takuhei MINAMI, Tokyo (JP); Hiroshi MITSUI, Tokyo (JP); Shohei ISHIDA, Tokyo (JP)

(73) Assignee: OLYMPUS MEDICAL SYSTEMS CORP., Tokyo (JP)

(21) Appl. No.: 18/372,166

(22) Filed: Sep. 25, 2023

Related U.S. Application Data
(60) Provisional application No. 63/409,849, filed on Sep. 26, 2022.

(51) Int. Cl. A61B 1/00

(52) U.S. Cl. CPC A61B 1/00126; A61B 1/00105; A61B 1/00103

(57) ABSTRACT

An endoscope connection mechanism includes a connection pipe sleeve including a hole having an axis, and a first optical shutter movable between a first state and a second state. The first optical shutter includes a first, a second and a third portions. The first portion includes a first end fixed to a base portion of the first optical shutter, and a second end provided distally relative to the first end. The second portion includes a third end connecting to the second end, and a fourth end provided distally relative to the third end. The third portion includes a fifth end connecting to the fourth end, and a sixth end provided distally relative to the fifth end. In the first state, the third portion is positioned to intersect the axis of the hole, and in the second state, the third portion is positioned not to intersect the axis of the hole.

### RELATED APPLICATION DATA

This application is based on and claims priority under 35 U.S.C. § 119 to U.S. Provisional Application No. 63/409,849 filed on Sep. 26, 2022, the entire contents of which are incorporated herein by reference.

### FIELD OF DISCLOSURE

The present disclosure relates to an endoscope connection mechanism configured to connect a light source device and an endoscope, an endoscope adapter configured to connect the light source device and the endoscope, and an endoscope system including the light source device and the endoscope.

### BACKGROUND

An endoscope is used for observation and the like of a region that cannot be seen from outside, by insertion of an elongated insertion section into a subject. The endoscope configures an endoscope system, together with a light source device configured to supply illumination light for illuminating the inside of the subject.

When a connector of the endoscope is connected to the light source device, the illumination light is guided to a distal end portion of the insertion section. The light source device is provided with a movable light-shielding member to prevent leakage of the illumination light to outside in a state where the connector is not connected.

Japanese Patent Application Laid-Open Publication No. 2002-136479 discloses a light source device including a light shielding spring configured to close an opening that is an optical path of illumination light. When a distal end of a connector of an endoscope pushes the light shielding spring, the light shielding spring opens and the illumination light is supplied to the endoscope.

Japanese Patent Application Laid-Open Publication No. 2012-70981 discloses a light source device where a light shielding plate is retracted from an optical path by a pin on a side surface of a connector of an endoscope.

These days, single-use endoscopes that are disposed of after being used once are being used. To connect a single-use endoscope to a light source device of a reusable endoscope that is used repeatedly, an adapter may be provided between the single-use endoscope and the light source device.

### SUMMARY

An endoscope connection mechanism of an embodiment is an endoscope connection mechanism that includes a connection pipe sleeve and a first optical shutter. The connection pipe sleeve includes a hole configured to receive a light guide, and having an axis. The first optical shutter is connected to the connection pipe sleeve, the first optical shutter is movable between a first state and a second state. The first optical shutter includes a first portion, a second portion and a third portion. The first portion includes a first end fixed to a base portion of the first optical shutter, and a second end provided distally relative to the first end. The second portion includes a third end connecting to the second end, and a fourth end provided distally relative to the third end. The third portion includes a fifth end connecting to the fourth end, and a sixth end provided distally relative to the fifth end. In the first state, the third portion is positioned to intersect the axis of the hole, and in the second state, the third portion is positioned not to intersect the axis of the hole.

An endoscope adapter of an embodiment comprises an endoscope adapter body including the endoscope connection mechanism. The endoscope adapter body is configured to connect an endoscope and an electrical appliance.

An endoscope system of an embodiment includes one or more of a light source device and an electrical appliance. The one or more of the light source device and the electrical appliance include the endoscope connection mechanism. The electrical appliance includes a light source device and a control device. The light source device is configured to supply an illumination light to the endoscope. The control device is configured to transmit and receive electrical signals to and from the endoscope.

### BRIEF DESCRIPTION OF THE DRAWINGS

FIG. 1 is a perspective view of an endoscope system of an embodiment.

FIG. 2 is a perspective view of main parts of the endoscope system of the embodiment.

FIG. 3 is an exploded perspective view of a connection mechanism of the embodiment.

FIG. 4 is a perspective view of an optical shutter of the connection mechanism of the embodiment.

FIG. 5A is a perspective view of the connection mechanism of the embodiment.

FIG. 5B is a cross-sectional view for describing operation of the connection mechanism of the embodiment.

FIG. 6 is a cross-sectional view for describing operation of the connection mechanism of the embodiment.

FIG. 7A is a perspective view of the connection mechanism of the embodiment.

FIG. 7B is a cross-sectional view for describing operation of the connection mechanism of the embodiment.

FIG. 8 is a plan view for describing a fabrication method of the optical shutter of the connection mechanism of the embodiment.

FIG. 9 is a cross-sectional view for describing operation of a connection mechanism of a first modification of the embodiment.

FIG. 10 is a configuration diagram of an endoscope system of a second modification of the embodiment.

FIG. 11 is a configuration diagram of an endoscope system of a third modification of the embodiment.

FIG. 12 is a configuration diagram of an endoscope system of a fourth modification of the embodiment.

FIG. 13 is an exploded perspective view of a connector of an endoscope of a reference example.

FIG. 14 is an exploded perspective view of a connector of an endoscope of a reference example.

FIG. 15 is an exploded perspective view of a connector of an endoscope of a reference example.

### DETAILED DESCRIPTION

Hereinafter, an embodiment of the present disclosure will be described with reference to the drawings. Note that the drawings based on the embodiment are schematic. A relationship between a thickness and a width of each part in a drawing, a ratio of respective thicknesses of parts, and the like are different from actual relationships. Dimensional relationships and ratios may be different between drawings. Illustration and assignment of reference signs may be omitted for some structural elements.

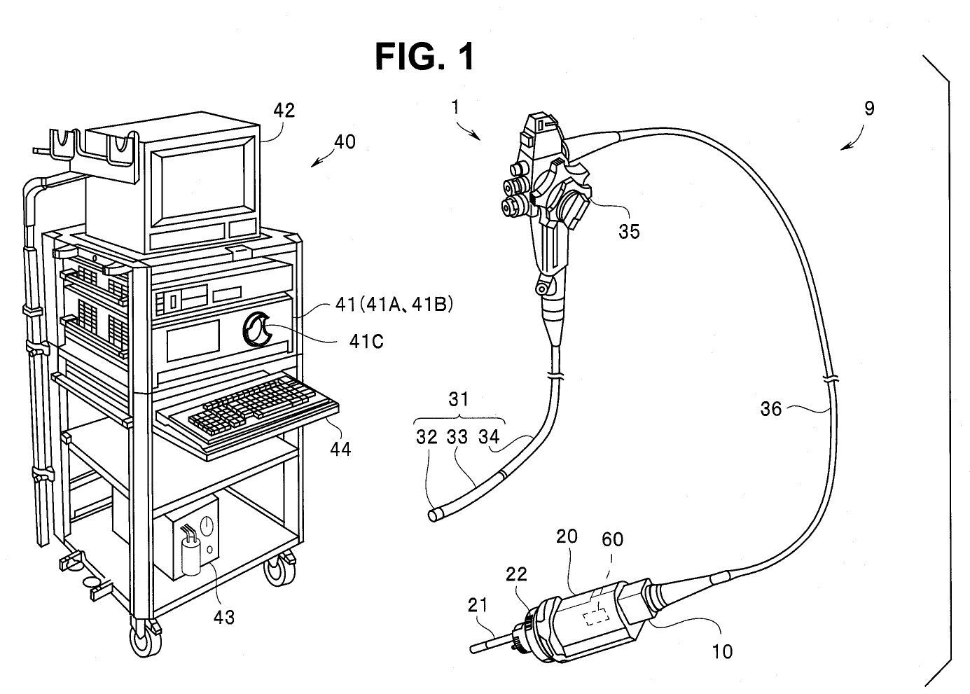

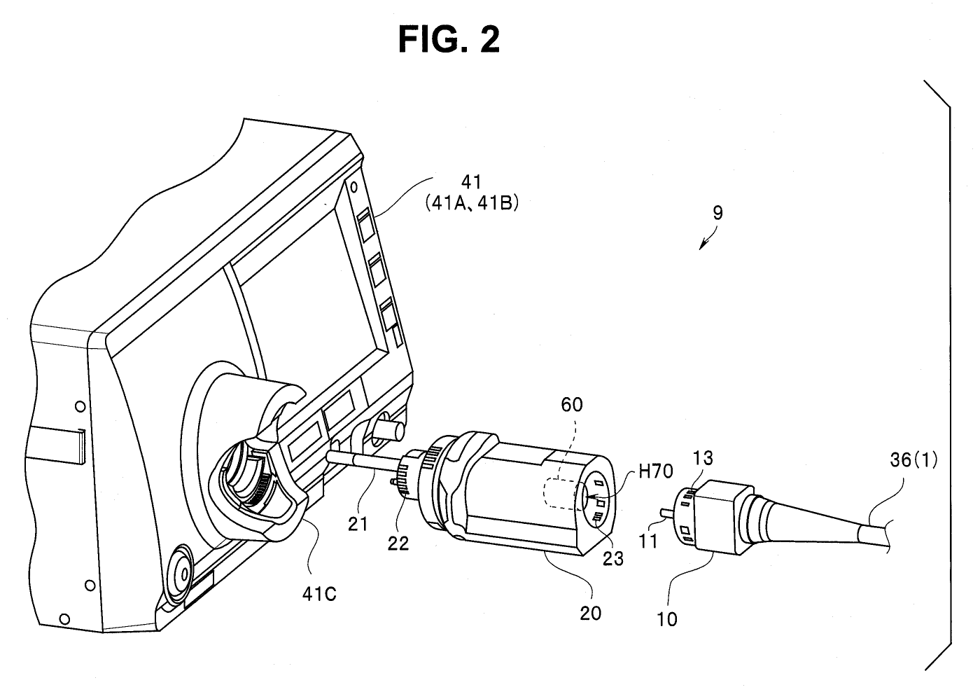

An endoscope system 9 of a present embodiment shown in FIGS. 1 and 2 includes an endoscope 1, an endoscope adapter 20 (hereinafter referred to as “adapter 20”), and a peripheral device unit 40 including a light source device 41A configured to supply illumination light. The endoscope adapter 20 can include an endoscope adapter body, the endoscope adapter body can include the endoscope connection mechanism 60. The endoscope adapter body is configured to connect the endoscope 1 and the electrical appliance 41.

An endoscope 1 includes an insertion section 31, an operation section 35, and a universal cord 36. The elongated insertion section 31 is inserted in a subject. The operation section 35 is provided at a proximal end of the insertion section 31. The universal cord 36 is provided extending from the operation section 35. A connector 10 is provided at a proximal end of the universal cord 36.

The operation section 35 includes a bending operation knob, and buttons for performing operations related to endoscope functions. The insertion section 31 includes, in the following order from a distal end side, a distal end portion 32, a bending portion 33, and a flexible tube 34. The distal end portion 32 includes an image pickup unit (not shown). The bending portion 33 that is freely bendable is provided at a proximal end of the distal end portion 32. The elongated flexible tube 36 is provided at a proximal end of the bending portion 33.

The peripheral device unit 40 includes a processor 41, a monitor 42, a gas supply device 43 as a fluid supply device, a keyboard 44 and the like that are mounted on a rack. The processor 41 is an electrical appliance integrating the light source device 41A and a control circuit (control device) 41B configured to transmit and receive electrical signals to and from the endoscope 1. The control circuit 41B controls the entire endoscope system 9, and also processes image signals that are outputted from the image pickup unit. An image processed by the processor 41 is displayed on the monitor 42. The keyboard 44 is used by a user to input data to the processor 41, for example. The gas supply device 43 supplies pressurized gas such as air, carbon dioxide, or nitrogen gas. Although not shown, the peripheral device unit 40 may include a water supply device configured to supply water to the endoscope 1. The electrical appliance 41 can include one or more of the light source device 41A and a control device 41B. The light source device 41A is configured to supply an illumination light to the endoscope 1. The control device 41B is configured to transmit and receive electrical signals to and from the endoscope 1. The one or more of the light source device 41A and the control device 42B can include endoscope connection mechanism 60.

The configuration of the endoscope system 9 described above is merely an example, and the endoscope system of the present disclosure is not limited to the configuration described above.

The endoscope 1 can be single-use endoscope that is disposed after being used once. The endoscope can include a single-use component and a multi-use component. The single-use endoscope or the single-use component can be disposed or returned to manufacturer of after being used once, and the multi-use component is a reusable component that is repeatedly used. A connection structure of the endoscope 1 to the processor 41 is different from the connection structure of a so-called reusable endoscope that is cleaned and used repeatedly. The adapter 20 is a relay adapter that is used to connect the processor 41 for a reusable endoscope and the connector 10 of the endoscope 1. When the single-use endoscope is connected to the light source device by the adapter, cost of the endoscope may be reduced because the connector 10 does not have to have a complex structure. Furthermore, because the adapter 20 is repeatedly used, cost per use of the endoscope system 9 including the adapter 20 is low. The endoscope adapter body can be a reusable product.

The adapter 20 includes a receptacle portion and a plug portion. The connector 10 of the endoscope 1 is inserted in the receptacle portion. The plug portion is inserted in a connector (receptacle) 41C of the processor 41. A light guide pipe sleeve 21, and a plurality of electric contacts 22 are disposed on the plug portion. Illumination light from the light source device 41A enters the light guide pipe sleeve 21. The electric contact 22 is connected to an electric contact of the processor 41. An endoscope connection mechanism 60 (hereinafter referred to as “connection mechanism 60”) configured to guide the illumination light to the connector 10, and a plurality of electric contacts 23 to be connected to electric contacts 13 of the connector 10 are disposed on the receptacle portion.

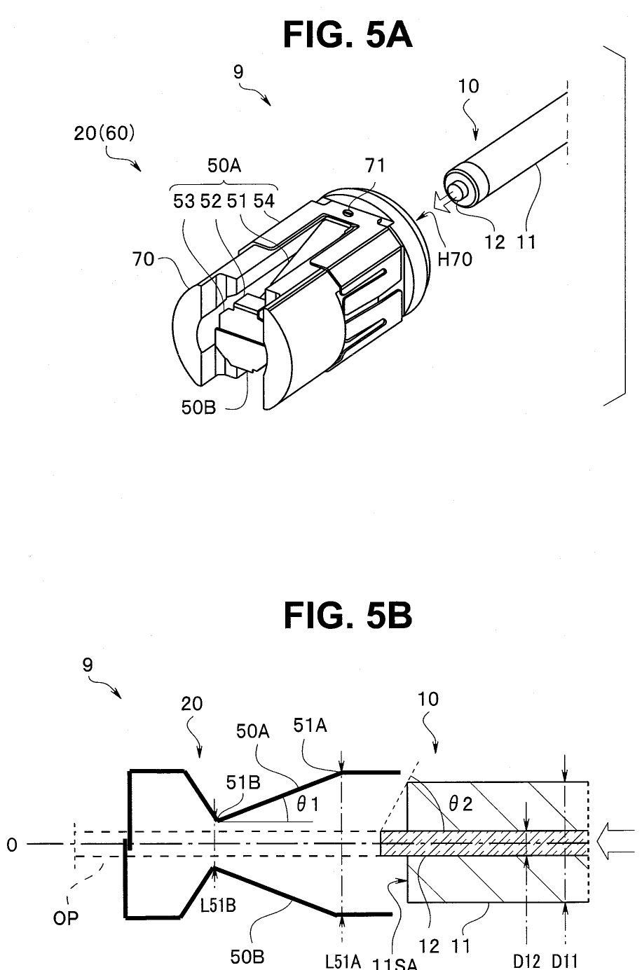

The plurality of electric contacts 13 are disposed on the connector 10. A light guide member (light guide) 12 such as a fiber bundle formed of a plurality of optical fibers is contained inside a light guide pipe sleeve (cylinder) 11 that is a cylinder of the connector 10 (see FIG. 5B and the like). The light guide member 12 may be fluorine-based resin or the like. The endoscope 1 includes the connector 10, the connector 10 includes the cylinder 11 inserting the light guide 12.

The illumination light from the light source device 41A is guided to the distal end portion 32 of the endoscope 1, via the connector 41C of the processor 41, the light guide pipe sleeve 21 of the adapter 20, and the light guide pipe sleeve 11 of the connector 10. Electrical signals are exchanged between the processor 41 and the endoscope 1 via the electric contact 22, the electric contact 23, and the electric contact 13.

Even when the light source device 41A includes a light shielding member for preventing leakage of the illumination light to outside, in a state where the endoscope is not connected to the adapter, the illumination light leaks to outside from the receptacle portion when the adapter is connected to the light source device 41A.

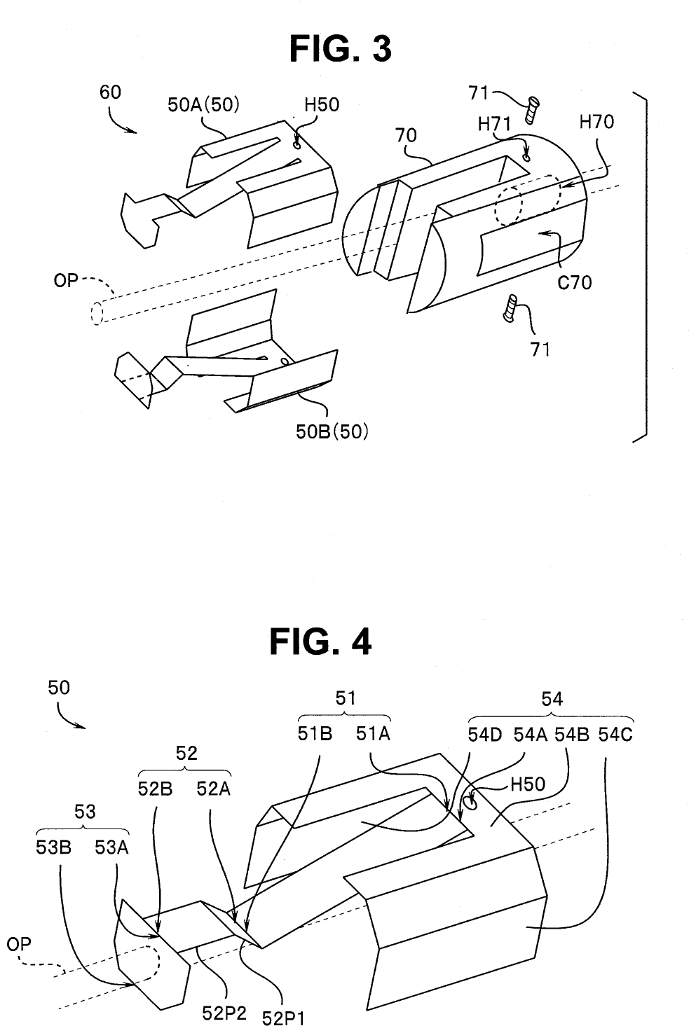

As shown in FIG. 3, the connection mechanism 60 of the present embodiment includes a connection pipe sleeve 70 including a hole H70 through which the light guide pipe sleeve 11 that is a cylinder is inserted and removed, a first optical shutter 50A, a second optical shutter 50B (hereinafter referred to as “optical shutter 50A, 50B”). The two optical shutters 50A, 50B that are disposed inside the hole H70 while facing each other across an optical path OP have a same shape. In the following, the two optical shutters 50A, 50B will each be referred to as an optical shutter 50. The hole H70 is configured to receive the light guide 12, the hole having an axis. The first optical shutter 50A is connected to the connection pipe sleeve 70, the first optical shutter 50A is movable between a first state and a second state. A shape of the first optical shutter 50A and a shape of the second optical shutter 50B can be the same. The second optical shutter 50B is movable between a third state and a fourth state. In the third state, the sixth portion 53 is positioned to intersect the axis of the hole H70, and in the fourth state, the sixth portion 53 is positioned not to intersect the axis of the hole H70. In the second state, the first portion 51 of the first optical shutter 50A does not come into contact with the light guide 11.

The adapter 20 including the optical shutters 50 is connected to the light source device 41A, but even in a state where the connector 10 is not connected, the illumination light is not leaked to outside. Furthermore, with the adapter 20, the illumination light is not leaked to outside even when the light source device 41A does not include a light shielding function.

The connection mechanism 60 includes a screw 71 that fixes the optical shutter 50 to the connection pipe sleeve 70. The screw 71 is inserted through a through hole H50 in the optical shutter 50, and is screwed into a hole H71 in the connection pipe sleeve 70. The screw 71 is inserted in the through hole H50 to fix the base portion 54 to the connection pipe sleeve 70. The optical shutter 50 may instead be fixed to the connection pipe sleeve 70 using an adhesive, sealing, filling, or the like. The base portion 54 of first optical shutter 50A is fixed to the connection pipe sleeve 70.

Furthermore, the connection pipe sleeve 70 includes a recess C70 where a part of the optical shutter 50 is to be fitted. Accordingly, with the connection mechanism 60, attachment of the optical shutter 50 to the connection pipe sleeve 70, and positioning of the optical shutter 50 relative to the connection pipe sleeve 70 are facilitated. The connection pipe sleeve 70 includes a recess C70, and a part of the base portion 54 is fitted to the recess C70. The recess C70 is on a surface of the connection pipe sleeve 70 that is different from a surface where the base portion 54 is fixed.

As shown in FIG. 4, the optical shutter 50 includes a first part (first portion) 51, a second part (second portion) 52, a third part (third portion) 53, and a fourth part (base portion) 54 that can be provided continuously. As described later, the first part 51, the second part 52, the third part 53, and the fourth part 54 are a cantilever leaf spring that is fabricated by subjecting one piece of flat sheet metal to a bending process. Due to the bending process, the first part 51, the second part 52, the third part 53, and the fourth part 54 are not on one flat surface. The first optical shutter 50A can be a leaf spring where the first portion 51, the second portion 52, and the third portion 53 are not coplanar.

The first part 51 includes a first end 51A that is a fixed end, and a second end 51B. The first portion 51 includes the first end 51A fixed to the base portion 54 of the first optical shutter 50A, and the second end 51B provided distally relative to the first end 51A. The second part 52 includes a third end 52A that is in contact with the second end 51B, and a fourth end 52B. The second portion 52 includes the third end 52A connecting to the second end 51B, and the fourth end 52B provided distally relative to the third end 52A. The third part 53 includes a fifth end 53A that is in contact with the fourth end 52B, and a sixth end 53B that is a free end. The third portion 53 includes the fifth end 53A connecting to the fourth end 52B, and the sixth end 53B provided distally relative to the fifth end 53A. The second end 51B and the third end 52A, and the fourth end 52B and the fifth end 53A are bent portions. In the first state, the third portion 53 is positioned to intersect the axis of the hole H70, and in the second state, the third portion 53 is positioned not to intersect the axis of the hole H70. The second end 51B of the first portion is positioned closer to the axis of the hole H70 than the first end 51A of the first portion. The third end 52A of the second portion 52 is positioned closer to the axis of the hole H70 than the fourth end 52B of the second portion 52. The sixth end 53B of the third portion 53 is positioned closer to the axis of the hole H70 than the fifth end of the third portion 53. In the first state, the second end 51B is positioned at a first radial position and, in the second state, the second end 51B is positioned at a second radial position. The second radial position is positioned farther from the axis of the hole H70 than the first radial position.

The second optical shutter 50B faces the first optical shutter 50A across the axis of the hole H70. The second optical shutter 50B is connected to the connection pipe sleeve 70. The second optical shutter 50B includes a fourth portion 51, a fifth portion 52 and a sixth portion 53. The fourth portion 51 includes a seventh end 51A fixed to a base portion 54 of the second optical shutter 50B, and an eighth end 51B provided distally relative to the seventh end 51A. The fifth portion 52 includes a ninth end 52A connecting to the eighth end 51B, and a tenth end 52B provided distally relative to the ninth end 52A. The sixth portion 53 includes an eleventh end 53A connecting to the tenth end 52B, and a twelfth end 53B provided distally relative to the eleventh end 53A. In the first state, the sixth portion is positioned to intersect the axis of the hole, and in the second state, the sixth portion is positioned not to intersect the axis of the hole.

The first end 51A, of the optical shutter 50, that is a fixed end may be a rigid plate that is fixed to the connection pipe sleeve 70 by a hinge, for example.

The first part 51 disposed in a periphery of the optical path OP is inclined relative to the optical path OP, and the second end 51B is disposed at a position closer to the optical path OP than the first end 51A. With respect to the first part 51, the second end 51B is disposed at a position closest to the optical path OP.

The second part 52 disposed in the periphery of the optical path OP includes a sloping surface 52P1 including the third end 52A, and a parallel surface 52P2 extending from the sloping surface 52P1 and including the fourth end 52B. The sloping surface 52P1 is inclined relative to the optical path OP. The parallel surface 52P2 is substantially parallel to the optical path OP. “Substantially parallel” includes a range of angles that have the same effect as when the parallel surface 52P2 and the optical path OP are parallel, or design deviation. With respect to the second part 52, the third end 52A is disposed at a position closest to the optical path OP.

With respect to the third part 53, the fifth end 53A is disposed at a position farther from an optical axis O of the optical path OP than the sixth end 53B. With respect to the third part 53, the fifth end 53A is disposed at a position farthest from the optical path OP. The third part 53 is a light shielding plate that shields the optical path. The sixth end 53B of the third portion 53 is positioned closer to the axis of the hole H70 than the second end 51B of the first portion 51.

The fourth part 54 is a retaining part for allowing the optical shutter 50 to be retained by the connection pipe sleeve 70. The fourth part 54 includes a seventh end 54A that is in contact with the first end 51A. The fourth part 54 includes a connection part 54B including the seventh end 54A, and a first fixing part 54C and a second fixing part 54D extending from the connection part 54B. The fourth part 54 is bent at a plurality of positions. Accordingly, the first fixing part 54C and the second fixing part 54D are disposed facing each other across the optical path OP. The connection part 54B, a part of the first fixing part 54C, and a part of the second fixing part 54D are positioned on different planes.

The first end 51A, the second end 51B, the third end 52A, the fourth end 52B, and the fifth end 53A of the optical shutter 50 may, but do not have to, have a same width W in a direction orthogonal to the optical path OP.

As shown in FIGS. 5A and 5B, in a state where the light guide pipe sleeve 11 of the connector 10 is not inserted in the hole H70 of the adapter 20 (a first state), the third part 53 of the optical shutter 50 is positioned on the optical path OP of the illumination light. Note that FIG. 5B does not show the connection pipe sleeve 70. The third part 53 of the optical shutter 50 is a light shielding plate that prevents the illumination light from leaking outside the adapter 20. The third part 53 that is the light shielding plate that is substantially orthogonal to the optical path OP has a higher light shielding performance than a light shielding plate that is inclined relative to the optical path OP. “Substantially orthogonal ” includes a range of angles that have the same effect as when the third part 53 and the optical path OP are orthogonal, or design deviation. A first gap is provided between the first end 51A of the first optical shutter 50A and the seventh end 51A of the second optical shutter 50B. A second gap is provided between the second end 51B of the first optical shutter 50A and the eighth end 51B of the second optical shutter 50B. An outer diameter of the cylinder 11 is smaller than the first gap, and the outer diameter of the cylinder 11 is greater than the second gap.

The third parts 53 of the two optical shutters 50 at least partially overlap each other on the optical path OP. Accordingly, the connection mechanism 60 has a high light shielding performance.

Note that an outer diameter D11 of the light guide pipe sleeve 11 that is a cylinder is smaller than a gap L51A between the first end 51A of the optical shutter 50A and the first end 51A of the optical shutter 50B. The outer diameter D11 is greater than a gap L51B between the second end 51B of the optical shutter 50A and the second end 51B of the optical shutter 50B. Moreover, an outer diameter D12 of the light guide member 12 is smaller than the gap L51B.

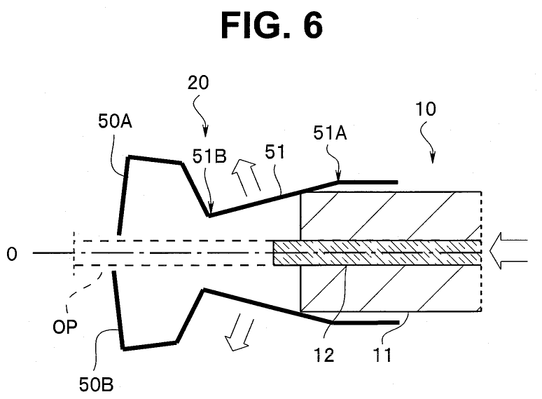

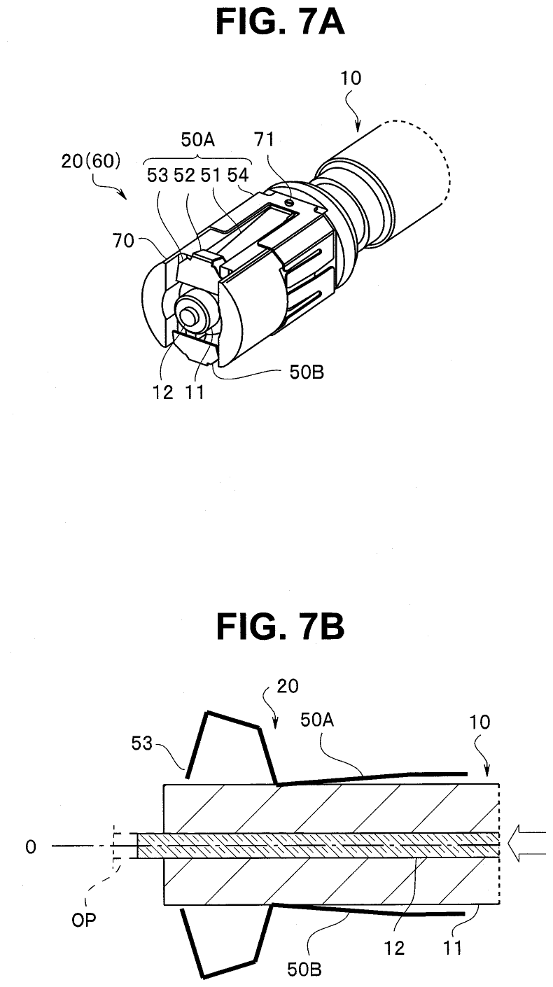

As shown in FIGS. 6, 7A and 7B, in a state where the light guide pipe sleeve 11 is inserted in the hole H70 of the adapter 20 (a second state), the first part 51 of the optical shutter 50 is pushed by the light guide pipe sleeve 11, and the second end 51B is moved in a direction of moving away from the optical path OP. Accordingly, the third part 53 moves out of the optical path OP. The third part 53 of the optical shutter 50A and the third part 53 of the optical shutter 50B retract from the optical path OP by moving in different directions. Because the third parts 53 are retracted from the optical path OP, the illumination light is guided to the light guide member 12 of the light guide pipe sleeve 11.

Note that with the connection mechanism 60, the optical shutter 50 is in contact with the light guide pipe sleeve 11 only at the first part 51. The third part 53 that is the light shielding plate does not come into contact with the light guide pipe sleeve 11.

The light guide member 12 protrudes from a distal end surface 11SA of the light guide pipe sleeve 11. The light guide member 12 is possibly damaged if the light guide member 12 comes into contact with the optical shutter.

As shown in FIG. 5B, with the endoscope system 9, in the first state before the light guide pipe sleeve 11 is inserted in the hole H70, a first angle θ1 that the first part 51 of the optical shutter 50 forms with the optical path OP is smaller than a second angle θ2 that a straight line joining a distal end outer circumference of the light guide pipe sleeve 11 and a distal end outer circumference of the light guide member 12 forms with the optical path OP. Accordingly, the light guide member 12 does not come into contact with the optical shutter 50. The first angle θ1 is defined between the first portion 51 and the axis of the hole H70. The second angle θ2 is defined between the straight line and the axis of the hole H70, the straight line connecting a distal end outer circumference of the cylinder 11 and a distal end outer circumference of the light guide 12. In the first state, the first angle θ1 is smaller than the second angle θ2.

The connection mechanism 60 of the present embodiment has a simple configuration, and also, the light guide member 12 does not come into contact with the optical shutter 50, and thus, the light guide member 12 is not damaged nor worn.

The light guide member 12 does not have to protrude from the distal end surface 11SA of the light guide pipe sleeve 11. However, the light guide member 12 that protrudes from the distal end surface 11SA of the light guide pipe sleeve 11 has higher light guiding efficiency than the light guide member 12 that does not protrude from the distal end surface 11SA of the light guide pipe sleeve 11.

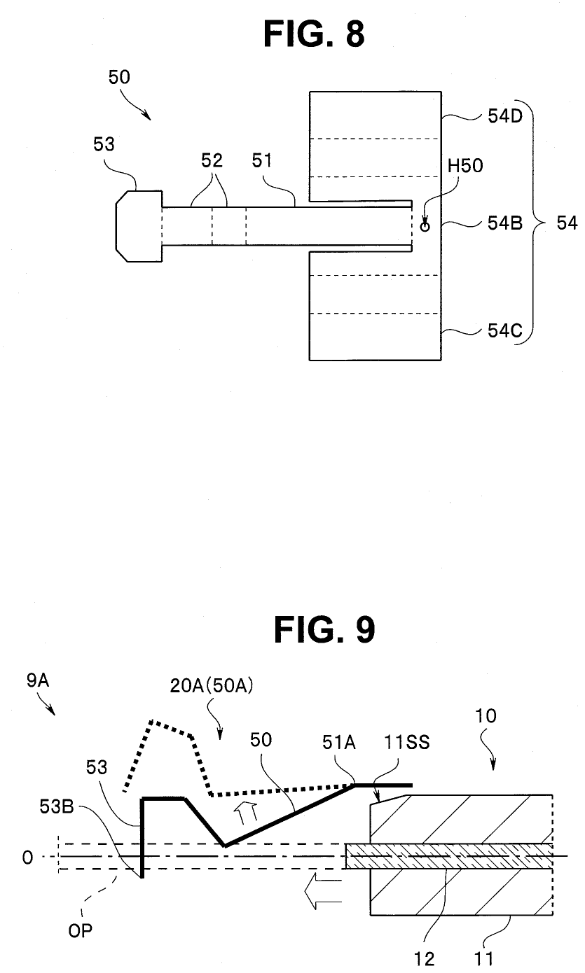

As described above, the first part 51, the second part 52, the third part 53, and the fourth part 54 of the optical shutter 50 are fabricated by subjecting one piece of sheet metal to a bending process (see FIG. 8). The sheet metal is made of stainless steel having a thickness of 0.5 mm, for example. Before processing, the first part 51, the second part 52, and the third part 53 are on a same plane. The first part 51, the second part 52, and the third part 53 of the optical shutter 50 after the bending process are not on the same plane.

The two optical shutters 50A, 50B that are disposed in an optical axis direction, at positions that are slightly separated, have a same configuration. The two optical shutters 50A, 50B may be manufactured by same steps, and thus, the connection mechanism 60 of the present embodiment enables a manufacturing cost to be reduced. Furthermore, with the connection mechanism 60 of the present embodiment, the two optical shutters 50A, 50B do not have to be distinguished from each other, and thus, assembly is facilitated.

### Modifications

A connection mechanism 60A and the like of a modification are similar to the connection mechanism 60 and the like of the embodiment, and achieve same advantageous effects. Accordingly, in the following description, structural elements having the same functions as the connection mechanism 60 and the like of the embodiment are denoted by same reference signs, and redundant description is omitted.

### First Modification

With an endoscope system 9A of a present modification shown in FIG. 9, the connection mechanism 60A includes only one optical shutter 50. The connection mechanism 60A of the modification has a simpler configuration than the connection mechanism 60 of the embodiment. Note that the third part 53 that is the light shielding plate of the optical shutter 50 of the connection mechanism 60A can be larger than the third part 53 of the connection mechanism 60 and can have a greater movement distance in a direction orthogonal to the optical axis.

It suffices if the connection mechanism of the present disclosure includes at least one optical shutter 50. The connection mechanism of the present disclosure may include three or more, such as four, optical shutters that are disposed at rotationally symmetric positions around the optical axis O.

With the connection mechanism 60A, a side surface 11SS of a distal end portion of the light guide pipe sleeve 11 is not parallel to the optical axis but is inclined relative to the optical axis. Accordingly, with the connection mechanism 60A, wear of the light guide pipe sleeve 11 due to contact with the optical shutter 50 is small. Also with the connection mechanism 60 of the embodiment, the side surface of the distal end portion of the light guide pipe sleeve 11 can be inclined.

### Second Modification

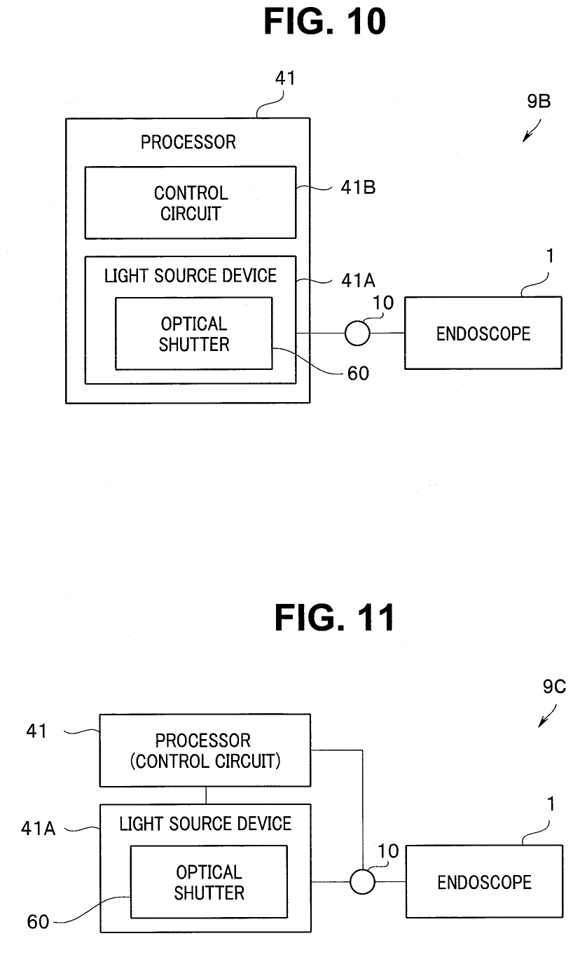

With an endoscope system 9B shown in FIG. 10, the connector 10 of the endoscope 1 is connected to the light source device 41A of the processor 41 without the adapter. The connection mechanism 60 including the optical shutter 50 is provided in the light source device 41A.

### Third Modification

With an endoscope system 9C shown in FIG. 11, the connector 10 of the endoscope 1 is connected to the light source device 41A that is a separate body from the processor 41 including a control circuit. The connection mechanism 60 including the optical shutter 50 is provided in a connector of the light source device 41A. A signal cable branching from the connector 10 of the endoscope 1 is connected to the processor 41 including the control circuit.

### Fourth Modification

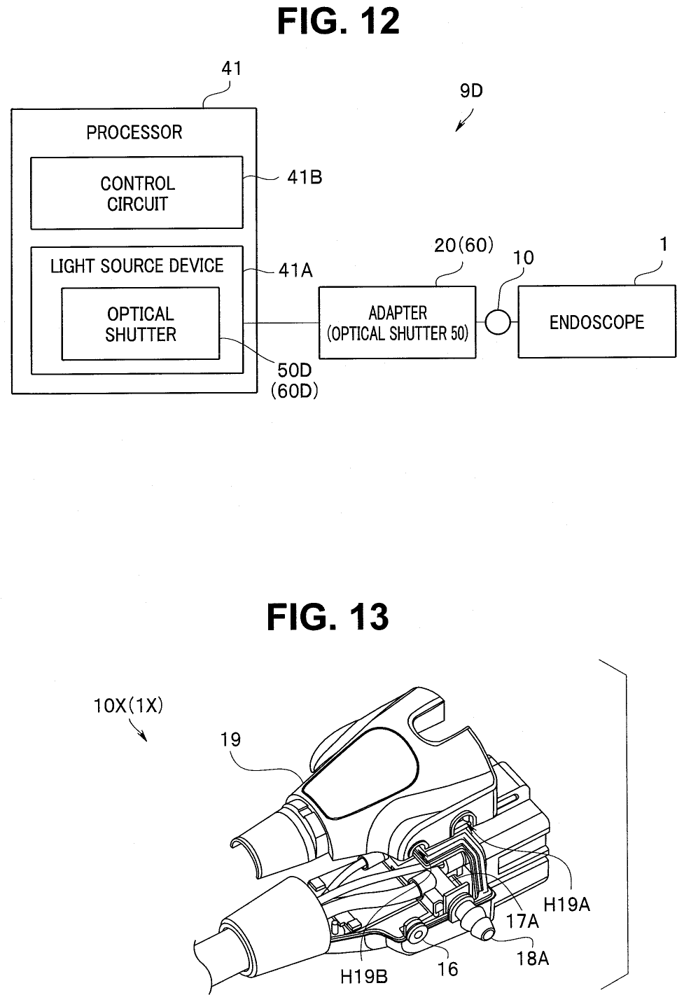

With an endoscope system 9D shown in FIG. 12, the adapter 20 includes the connection mechanism 60 including at least one optical shutter 50. Furthermore, a connection mechanism 60D including at least one third optical shutter 50D for preventing leakage of the illumination light to outside is provided in the light source device 41A. The third optical shutter 50D may have a substantially same configuration as the optical shutter 50A.

With the endoscope system 9D, the light source device 41A and a control circuit 41B are housed in one housing as the processor 41. However, also with the endoscope system 9D, as with the endoscope system 9C, the light source device 41A may be a separate body from the processor 41 including the control circuit 41B.

The endoscope 1 is a medical flexible endoscope, but an endoscope in a different embodiment may be an industrial endoscope, or may be a rigid endoscope including a straight rigid tube instead of the flexible tube 36. Moreover, the endoscope 1 of the embodiment and the modifications may be a reusable endoscope that is repeatedly used by being subjected to a cleaning/disinfecting process after being used.

### Reference Example

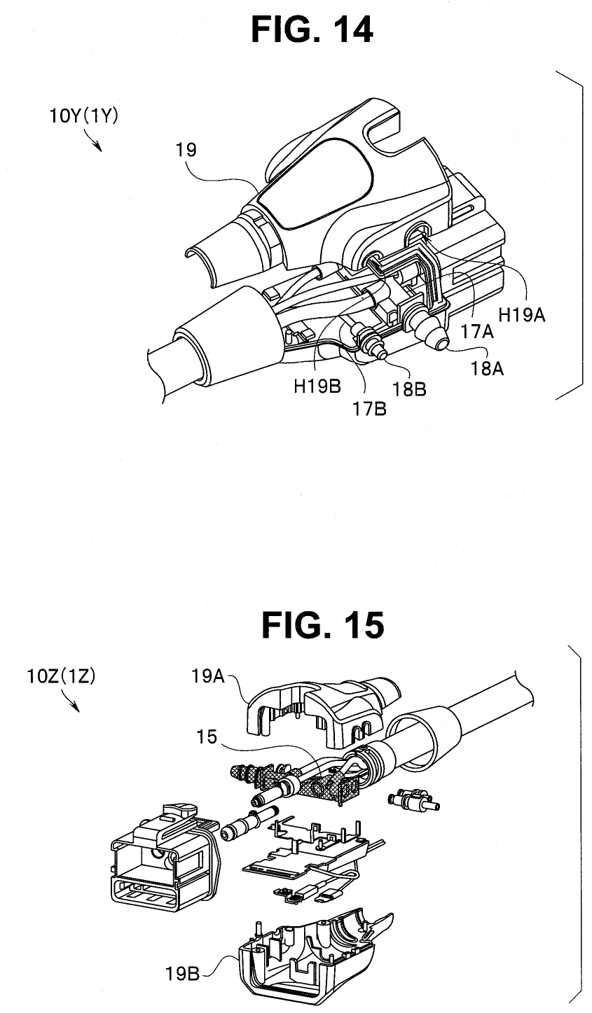

A housing (an external component) 19 of a connector 10Y of an endoscope 1X shown in FIG. 13 and the housing 19 of a connector 10Y of an endoscope 1Y shown in FIG. 14 both include two through holes H19A, H19B. With the endoscope 1X, a pipe sleeve 18A is disposed in the through hole H19A. The pipe sleeve 18A is a part of a molded conduit 17A. A plug 16 made of rubber or resin is fitted in the through hole H19B.

In contrast, with the endoscope 1Y shown in FIG. 14, the pipe sleeve 18A is disposed in the through hole H19A, as in the case of the endoscope 1X. An additional pipe sleeve 18B is disposed in the through hole H19B. A tube 17B is connected to the additional pipe sleeve 18B.

With the endoscope lx, the through hole H19B is an unused, useless hole. However, the housing 19 including the through hole H19B may be used for any of the endoscope lx and the endoscope 1Y. When a component such as the housing can be configured a common component of endoscopes of different specifications, a manufacturing cost of the endoscope may be reduced.

A housing of the adapter for connecting the endoscope and the processor can be also configured a common component of adapters of different specifications.

A connector 10Z of an endoscope 1Z of a reference example shown in FIG. 15 includes a molded conduit 15 formed by resin-molding a plurality of conduits made of resin. The connector 10Z is fabricated by sandwiching internal components such as the molded conduit 15 and the like by external components 19A, 19B that are divided into two. The connector 10Z including the molded conduit 15 can be more easily assembled than a connector including a tube.

Although not shown, a conventional connector includes an aluminum die-cast fixing plate for fixing the tube and the like, for example.

The connector 10Z of the reference example does not include the fixing plate, and is thus more easily manufactured and is less expensive than the conventional connector.

The configuration of the connector 10Z of the reference example can be adopted also for the housing of the adapter that connects the endoscope and the processor.

The present disclosure is not limited to the embodiment explained above. Various changes, alterations, and the like are possible within a range not changing the gist of the disclosure.

The embodiment of the present disclosure is aimed at providing an endoscope connection mechanism with which leakage of light is prevented by a simple configuration that does not damage a light guide member disposed in a connector of an endoscope, an endoscope adapter including the connection mechanism with which leakage of light is prevented by a simple configuration that does not damage the light guide member disposed in the connector of the endoscope, and an endoscope system including the connection mechanism with which leakage of light is prevented by a simple configuration that does not damage the light guide member disposed in the connector of the endoscope.

According to the embodiment of the present disclosure, there may be provided an endoscope connection mechanism with which leakage of light is prevented by a simple configuration that does not damage a light guide member disposed in a connector of an endoscope, an endoscope adapter including the connection mechanism with which leakage of light is prevented by a simple configuration that does not damage the light guide member disposed in the connector of the endoscope, and an endoscope system including the connection mechanism with which leakage of light is prevented by a simple configuration that does not damage the light guide member disposed in the connector of the endoscope.

Example 1. An endoscope connection mechanism that connects a light source device for supplying illumination light to an endoscope and a connector of the endoscope, the endoscope connection mechanism comprising:
a connection pipe sleeve including a hole through which a cylinder of the connector is inserted and removed, the cylinder containing a light guide member; and
at least one optical shutter provided inside the hole, wherein
the optical shutter includes a first part, a second part, and a third part, the first part including a first end that is a fixed end, and a second end, the second part including a third end that is in contact with the second end, and a fourth end, the third part including a fifth end that is in contact with the fourth end, and a sixth end that is a free end,
in a state where the cylinder is not inserted in the hole, the third part is positioned on an optical path of the illumination light, and
in a state where the cylinder is inserted in the hole, the first part is pushed by the cylinder, and the third part is positioned outside the optical path.

Example 2. The endoscope connection mechanism according to Example 1, wherein, with respect to the first part, the second end is disposed at a position closer to the optical path than the first end.

Example 3. The endoscope connection mechanism according to Example 1, wherein, when the first part is pushed by the cylinder, the second end is moved in a direction of moving away from the optical path.

Example 4. The endoscope connection mechanism according to Example 1, wherein the optical shutter is a cantilever leaf spring where the first part, the second part, and the third part are not positioned on a same plane.

Example 5. The endoscope connection mechanism according to Example 1, wherein, with respect to the second part, the third end is disposed at a position closer to the optical path than the fourth end.

Example 6. The endoscope connection mechanism according to Example 1, wherein
the second part includes a sloping surface including the third end, and a parallel surface extending from the sloping surface and including the fourth end, and
in a state where the cylinder is not inserted in the hole, the parallel surface is disposed substantially in parallel with the optical path.

Example 7. The endoscope connection mechanism according to Example 1, wherein, with respect to the third part, the fifth end is disposed at a position that is farther away from an optical axis of the optical path than the sixth end.

Example 8. The endoscope connection mechanism according to Example 1, wherein, in a state where the cylinder is not inserted in the hole, the third part is disposed substantially perpendicular to the optical path.

Example 9. The endoscope connection mechanism according to Example 1, wherein the first part, the second part, and the third part are formed of one piece of sheet metal that is bent.

Example 10. The endoscope connection mechanism according to Example 1, wherein the first end, the second end, the third end, the fourth end, and the fifth end of the optical shutter have a same width in a direction orthogonal to the optical path.

Example 11. The endoscope connection mechanism according to Example 1, wherein the first part does not come into contact with the light guide member even in a state where the cylinder is inserted in the hole.

Example 12. The endoscope connection mechanism according to Example 1, wherein, in a state where the cylinder is not inserted in the hole, a first angle that the first part forms with the optical path is smaller than a second angle that a straight line joining a distal end outer circumference of the cylinder and a distal end outer circumference of the light guide member forms with the optical path.

Example 13. The endoscope connection mechanism according to Example 1, wherein
the optical shutter further includes a fourth part including a seventh end that is in contact with the first end,
the fourth part includes a connection part including the seventh end, and a first fixing part and a second fixing part extending from the connection part, and
the first fixing part and the second fixing part are disposed facing each other across the optical path.

Example 14. The endoscope connection mechanism according to Example 13, wherein a part of the fourth part is positioned on a plane different from the connection part.

Example 15. The endoscope connection mechanism according to Example 13, comprising a screw configured to fix the fourth part to the connection pipe sleeve by being inserted in a through hole in the connection part.

Example 16. The endoscope connection mechanism according to Example 13, wherein the connection pipe sleeve includes a recess that allows a part of the fourth part to be fitted.

Example 17. The endoscope connection mechanism according to Example 16, wherein the connection pipe sleeve includes the recess on a surface that is different from a surface where the connection part of the fourth part is fixed.

Example 18. The endoscope connection mechanism according to Example 13, wherein the first part, the second part, the third part, and the fourth part are formed of one piece of sheet metal that is bent.

Example 19. The endoscope connection mechanism according to Example 1, comprising a first optical shutter and a second optical shutter that are disposed facing each other across the optical path.

Example 20. The endoscope connection mechanism according to Example 19, wherein the first optical shutter and the second optical shutter have a same shape.

Example 21. The endoscope connection mechanism according to Example 19, wherein an outer diameter of the cylinder is smaller than a gap between the first end of the first optical shutter and the first end of the second optical shutter, and is greater than a gap between the second end of the first optical shutter and the second end of the second optical shutter.

Example 22. The endoscope connection mechanism according to Example 1, wherein the endoscope connection mechanism is provided in an adapter configured to relay between the light source device and the connector of the endoscope.

Example 23. The endoscope connection mechanism according to Example 22, wherein the endoscope is a single-use endoscope that is disposed of after being used once.

Example 24. The endoscope connection mechanism according to Example 1, wherein the endoscope connection mechanism is provided in the light source device.

Example 25. The endoscope connection mechanism according to Example 1, wherein the endoscope connection mechanism is provided in an electrical appliance integrating the light source device configured to supply the illumination light to the endoscope, and a control device configured to transmit and receive electrical signals to and from the endoscope.

Example 26. The endoscope connection mechanism according to Example 1, wherein the endoscope connection mechanism is provided in each of the light source device, and an adapter configured to relay between the light source device and the connector of the endoscope.

Example 27. An endoscope adapter that connects a light source device for supplying illumination light to an endoscope and a connector of the endoscope, the endoscope adapter comprising:
a connection pipe sleeve including a hole through which a cylinder of the connector is inserted and removed, the cylinder containing a light guide member; and
at least one optical shutter provided inside the hole, wherein
the optical shutter includes a first part, a second part, and a third part, the first part including a first end that is a fixed end, and a second end, the second part including a third end that is in contact with the second end, and a fourth end, the third part including a fifth end that is in contact with the fourth end, and a sixth end that is a free end,
in a state where the cylinder is not inserted in the hole, the third part is positioned on an optical path of the illumination light, and
in a state where the cylinder is inserted in the hole, the third part is positioned outside the optical path due to the first part being pushed and moved by the cylinder.

Example 28. The endoscope adapter according to Example 27, comprising a first optical shutter and a second optical shutter that are disposed facing each other across the optical path.

Example 29. The endoscope adapter according to Example 27, wherein the first part, the second part, and the third part of the optical shutter are formed of one piece of sheet metal.

Example 30. The endoscope adapter according to Example 27, wherein
the endoscope is a single-use endoscope that is disposed of after being used once, and
the endoscope adapter performs relaying between the light source device compatible with a reusable endoscope and the endoscope.

Example 31. The endoscope adapter according to Example 27, wherein the endoscope adapter is a reusable product that is used repeatedly.

Example 32. An endoscope system comprising:
an endoscope including a connector, the connector including a cylinder containing a light guide member; and
an adapter configured to connect a light source device that supplies illumination light to the endoscope and the connector, the adapter including a connection pipe sleeve including a hole through which the cylinder is inserted and removed, and at least one optical shutter provided inside the hole, wherein
the optical shutter includes a first part, a second part, and a third part, the first part including a first end that is a fixed end, and a second end, the second part including a third end that is in contact with the second end, and a fourth end, the third part including a fifth end that is in contact with the fourth end, and a sixth end that is a free end,
in a state where the cylinder is not inserted in the hole, the third part is positioned on an optical path of the illumination light, and
in a state where the cylinder is inserted in the hole, the third part is positioned outside the optical path due to the first part being pushed and moved by the cylinder.

Example 33. The endoscope system according to Example 32, wherein the first part of the optical shutter does not come into contact with the light guide member.

Example 34. The endoscope system according to Example 32, wherein a side surface of a distal end portion of the cylinder is inclined.

Example 35. The endoscope system according to Example 32, wherein a distal end surface of the light guide member protrudes from a distal end surface of the cylinder.

Example 36. The endoscope system according to Example 32, comprising a first optical shutter and a second optical shutter, wherein
the first optical shutter and the second optical shutter have a same shape, and
the first optical shutter and the second optical shutter are disposed facing each other across the optical path.

Example 37. The endoscope system according to Example 32, wherein the light source device includes a third optical shutter configured to prevent leakage of the illumination light to outside in a state where the adapter is not connected.

Example 38. The endoscope connection mechanism according to Example 1, comprising the at least one optical shutter that are disposed facing each other across the optical path.

What is claimed is:

1. An endoscope connection mechanism, comprising:
a connection pipe sleeve including a hole configured to receive a light guide, the hole having an axis; and
a first optical shutter connected to the connection pipe sleeve, the first optical shutter movable between a first state and a second state,
wherein the first optical shutter includes:
a first portion including a first end fixed to a base portion of the first optical shutter, and a second end provided distally relative to the first end,
a second portion including a third end connecting to the second end, and a fourth end provided distally relative to the third end, and
a third portion including a fifth end connecting to the fourth end, and a sixth end provided distally relative to the fifth end,
wherein, in the first state, the third portion is positioned to intersect the axis of the hole, and
wherein, in the second state, the third portion is positioned not to intersect the axis of the hole.

2. The endoscope connection mechanism according to claim 1, wherein the second end of the first portion is positioned closer to the axis of the hole than the first end of the first portion,
wherein the third end of the second portion is positioned closer to the axis of the hole than the fourth end of the second portion, and
wherein the sixth end of the third portion is positioned closer to the axis of the hole than the fifth end of the third portion.

3. The endoscope connection mechanism according to claim 1, wherein, in the first state, the second end is positioned at a first radial position and, in the second state, the second end is positioned at a second radial position, and
wherein the second radial position is positioned farther from the axis of the hole than the first radial position.

4. The endoscope connection mechanism according to claim 1, wherein the first optical shutter is a leaf spring where the first portion, the second portion, and the third portion are not coplanar.

5. The endoscope connection mechanism according to claim 1, wherein the sixth end of the third portion is positioned closer to the axis of the hole than the second end of the first portion.

6. The endoscope connection mechanism according to claim 1, wherein the base portion of first optical shutter is fixed to the connection pipe sleeve.

7. The endoscope connection mechanism according to claim 6, further comprising a screw, wherein the screw is inserted in a through hole to fix the base portion to the connection pipe sleeve.

8. The endoscope connection mechanism according to claim 6, wherein the connection pipe sleeve includes a recess, and wherein a part of the base portion is fitted to the recess.

9. The endoscope connection mechanism according to claim 8, wherein the recess is on a surface of the connection pipe sleeve that is different from a surface where the base portion is fixed.

10. The endoscope connection mechanism according to claim 1, further comprising a second optical shutter facing the first optical shutter across the axis of the hole,
wherein the second optical shutter includes:
a fourth portion including a seventh end fixed to a base portion of the second optical shutter, and an eighth end provided distally relative to the seventh end,
a fifth portion including a ninth end connecting to the eighth end, and a tenth end provided distally relative to the ninth end, and
a sixth portion including an eleventh end connecting to the tenth end, and a twelfth end provided distally relative to the eleventh end,
wherein, in the first state, the sixth portion is positioned to intersect the axis of the hole, and
wherein, in the second state, the sixth portion is positioned not to intersect the axis of the hole.

11. The endoscope connection mechanism according to claim 1, further comprising a second optical shutter connected to the connection pipe sleeve,
wherein a shape of the first optical shutter and a shape of the second optical shutter are the same, and
wherein the second optical shutter faces the first optical shutter across the axis of the hole.

12. The endoscope connection mechanism according to claim 11, wherein, the second optical shutter movable between a third state and a fourth state,
wherein, in the third state, the sixth portion is positioned to intersect the axis of the hole, and
wherein, in the fourth state, the sixth portion is positioned not to intersect the axis of the hole.

13. An endoscope adapter, comprising:
an endoscope adapter body including the endoscope connection mechanism according to claim 1,
wherein the endoscope adapter body is configured to connect an endoscope and an electrical appliance.

14. The endoscope adapter according to claim 13, wherein the endoscope adapter body is a reusable product.

15. An endoscope system, comprising:
an endoscope including a connector, the connector including a cylinder inserting the light guide; and
the endoscope connection mechanism according to claim 1.

16. The endoscope system according to claim 15, wherein, in the second state, the first portion of the first optical shutter does not come into contact with the light guide.

17. The endoscope system according to claim 15, wherein a first angle is defined between the first portion and the axis of the hole,
wherein a second angle is defined between a straight line and the axis of the hole, the straight line connecting a distal end outer circumference of the cylinder and a distal end outer circumference of the light guide, and
wherein, in the first state, the first angle is smaller than the second angle.

18. The endoscope system according to claim 15, further comprising a second optical shutter facing the first optical shutter across the axis of the hole,
wherein the second optical shutter includes:
a fourth portion including a seventh end fixed to a base portion of the second optical shutter, and an eighth end provided distally relative to the seventh end,
a fifth portion including a ninth end connecting to the eighth end, and a tenth end provided distally relative to the ninth end, and
a sixth portion including an eleventh end connecting to the tenth end, and a twelfth end provided distally relative to the eleventh end,
wherein, in the first state, the sixth portion is positioned to intersect the axis of the hole, and
wherein, in the second state, the sixth portion is positioned not to intersect the axis of the hole,
wherein, in the first state, a gap is provided between the first end of the first optical shutter and the seventh end of the second optical shutter, and wherein, in the first state, an outer diameter of the cylinder is smaller than the gap.

19. The endoscope system according to claim 15, wherein the endoscope is a single-use endoscope.

20. An endoscope system, comprising:
an electrical appliance including one or more of a light source device and a control device,
wherein the light source device is configured to supply an illumination light to the endoscope,
wherein the control device is configured to transmit and receive electrical signals to and from the endoscope, and
wherein the one or more of the light source device and the control device include endoscope connection mechanism according to claim 1.
